(19) United States
(12) Patent Application Publication
(10) Pub. No.: US 2025/0082247 A1
(43) Pub. Date: Mar. 13, 2025

(54) SYSTEM FOR ASSESSING CARDIAC CONDITION

(71) Applicants: Inventec (Pudong) Technology Corporation, Shanghai (CN); INVENTEC CORPORATION, TAIPEI CITY (TW)

(72) Inventors: Sergio GONZALEZ-VAZQUEZ, TAIPEI CITY (TW); Wei-Chao CHEN, TAIPEI CITY (TW)

(21) Appl. No.: 18/414,430

(22) Filed: Jan. 16, 2024

(30) Foreign Application Priority Data
Sep. 11, 2023 (CN) 202311169291.2

(51) Int. Cl. A61B 5/35; A61B 5/024; A61B 5/28; A61B 5/308; A61B 5/352; A61B 5/353; G16H 50/30

(52) U.S. Cl. CPC A61B 5/35; A61B 5/02416; A61B 5/28; A61B 5/308; A61B 5/352; A61B 5/353; G16H 50/30

(57) ABSTRACT

A system for assessing a cardiac condition includes an electrocardiogram (ECG) sensor, a photoplethysmography (PPG) sensor, and a processing circuit. The ECG sensor obtains an ECG signal related to a user. The PPG sensor obtains a PPG signal related to the user. The processing circuit generates a cardiac assessment result based on the PPG signal sensed during a first time period and the ECG signal sensed during a second time period. The first time period is longer than the second time period.

### RELATED APPLICATION

This application claims priority to China Application Serial Number 202311169291.2 filed Sep. 11, 2023, which is herein incorporated by reference.

### BACKGROUND

### Field of Invention

The present disclosure relates to a system for assessing a heart condition based on machine learning models.

### Description of Related Art

Conventional techniques for assessing heart failure require a combination of the patient's medical history, interviews, and related tests, all of which come at a significant cost. To address this issue, there are now many wearable devices available on the market to capture electrocardiogram (ECG) signals from the patients. Furthermore, machine learning technologies are becoming increasingly prevalent, and ECG-based machine learning methods can already provide satisfactory assessment results. However, these methods typically involve capturing relatively short (for example, tens of seconds) ECG signals, lacking a comprehensive assessment of the heart's condition over a longer period. Obtaining long-term ECG signals faces many challenges, such as electrodes for sensing the electrocardiogram being unable to maintain prolonged contact with the patient's skin, as well as issues related to battery life.

### SUMMARY

Embodiment of the present disclosure provide a system including an electrocardiogram (ECG) sensor configured to obtain an ECG signal related to a user, a photoplethysmography (PPG) sensor configured to obtain a PPG signal related to the user, and a processing circuit electrically connected to the electrocardiogram sensor and the photoplethysmography sensor. The processing circuit is configured to generate a cardiac assessment result according to the PPG signal sensed during a first time period and the ECG signal sensed during a second time period, in which the first time period is longer than the second time period.

In some embodiments, the processing circuit is configured to input the ECG signal into a first machine learning model to generate a first feature vector. The processing circuit is configured to detect multiple beats from the PPG signal, calculate multiple beat intervals according to the beats, and input the beat intervals into a second machine learning model to generate a second feature vector. The processing circuit is configured to input the first feature vector and the second feature vector into a third machine learning model to generate the cardiac assessment result.

In some embodiments, the first time period includes multiple sub-periods, the processing circuit is configured to select the beats sensed in a time segment from each of the sub-periods, and calculate the beat intervals according to the selected beats.

In some embodiments, the first machine learning model is a convolutional neural network, the second machine learning model is a transformer, and the third machine learning model is a multilayer perceptron network.

In some embodiments, the processing circuit is configured to recognize multiple R peaks in the ECG signal, calculate multiple RR intervals among the R peaks, and calculate multiple ECG features according to the RR intervals. The processing circuit is configured to detect multiple beats from the PPG signal, calculate multiple beat intervals according to the beats, and calculate multiple PPG features according to the beat intervals. The processing circuit is configured to input the ECG features and the PPG features into a machine learning model to generate the cardiac assessment result.

In some embodiments, the processing circuit is configured to recognize multiple cardiac cycles from the ECG signal, and align the cardiac cycles based on the R peaks to generate a template cycle. The processing circuit is configured to recognize a template P peak, a template Q peak, a template R peak, a template S peak, and a template T peak in the template cycle. The processing circuit is configured to calculate a time difference between the template R peak and one of the template P peak, the template Q peak, the template S peak and the template T peak as one of the ECG features.

In some embodiments, the processing circuit is configured to calculate an amplitude difference between a baseline amplitude and one of the template P peak, the template Q peak, the template R peak, the template S peak, and the template T peak as one of the ECG features.

In some embodiments, the first time period includes multiple sub-periods, the processing circuit is configured to select the beats sensed in a time segment from each of the sub-periods, and concatenate the selected beats to calculate the beat intervals.

In some embodiments, the cardiac assessment result includes a survival curve and a risk score, and the survival curve includes multiple probabilities of not experiencing a heart failure on a time axis.

In some embodiments, the processing circuit is configured to generate the risk score according to the probability at a predetermined time on the time axis.

### BRIEF DESCRIPTION OF THE DRAWINGS

The invention can be more fully understood by reading the following detailed description of the embodiment, with reference made to the accompanying drawings as follows.

FIG. 1 is a schematic diagram illustrating an assessment system for cardiac conditions according to an embodiment.

FIG. 2 is a flowchart illustrating the process of generating the cardiac assessment result according to the first embodiment.

FIG. 3 illustrates the processing of the PPG signal according to one embodiment.

FIG. 4 is a schematic diagram illustrating the cardiac assessment result according to the first embodiment.

FIG. 5 is a flowchart illustrating the generation of a cardiac assessment result according to the second embodiment.

FIG. 6 is a schematic diagram illustrating the calculation of the ECG features according to the second embodiment.

### DETAILED DESCRIPTION

Specific embodiments of the present invention are further described in detail below with reference to the accompanying drawings, however, the embodiments described are not intended to limit the present invention and it is not intended for the description of operation to limit the order of implementation. Moreover, any device with equivalent functions that is produced from a structure formed by a recombination of elements shall fall within the scope of the present invention. Additionally, the drawings are only illustrative and are not drawn to actual size.

The using of “first”, “second”, “third”, etc. in the specification should be understood for identifying units or data described by the same terminology, but are not referred to particular order or sequence.

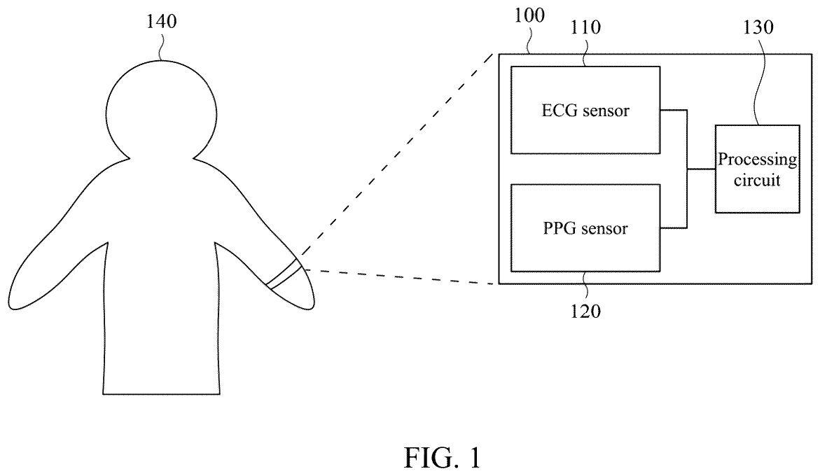

FIG. 1 is a schematic diagram illustrating an assessment system for cardiac conditions according to an embodiment. Referring to FIG. 1, in this embodiment, the assessment system 100 is implemented as a wearable device, such as a smartwatch or wristband, worn by a user 140. The assessment system 100 includes an electrocardiogram (ECG) sensor 110, a photoplethysmography (PPG) sensor 120, and a processing circuit 130. The electrocardiogram sensor 110 is used to acquire an ECG signal of the user 140. The PPG sensor 120 is used to acquire a PPG signal of the user 140. The PPG signal is based on a light source and a light sensor, and does not require direct contact with the user 140, offering the advantage of relatively low power consumption and thus is suitable for long-term continuous monitoring. In this embodiment, the PPG signal is sensed over a first time period and the ECG signal is sensed over a second time period. The first time period is longer than the second time period. For example, the first time period may be several hours or multiple days, while the second time period may be several tens of seconds. As such, the PPG signal can provide more comprehensive information over a longer period, while the ECG provides relatively more accurate cardiac information. Next, the processing circuit 130 executes one or more machine learning algorithms based on the PPG signal and ECG signal to generate a cardiac assessment result. This cardiac assessment result may include a risk score, which can be presented to the user 140 in various forms such as imagery, voice, or text to indicate the likelihood of heart failure. In other embodiments, the cardiac assessment result may also include abnormal segments of the ECG, timestamps of irregular heart rates, or an assessment report based on this information. The disclosure does not limit the content or presentation format of the cardiac assessment result (it could be in the form of imagery, voice, text, or digital data, etc.). Below are two embodiments illustrating how to use machine learning models to generate the cardiac assessment result.

### First Embodiment

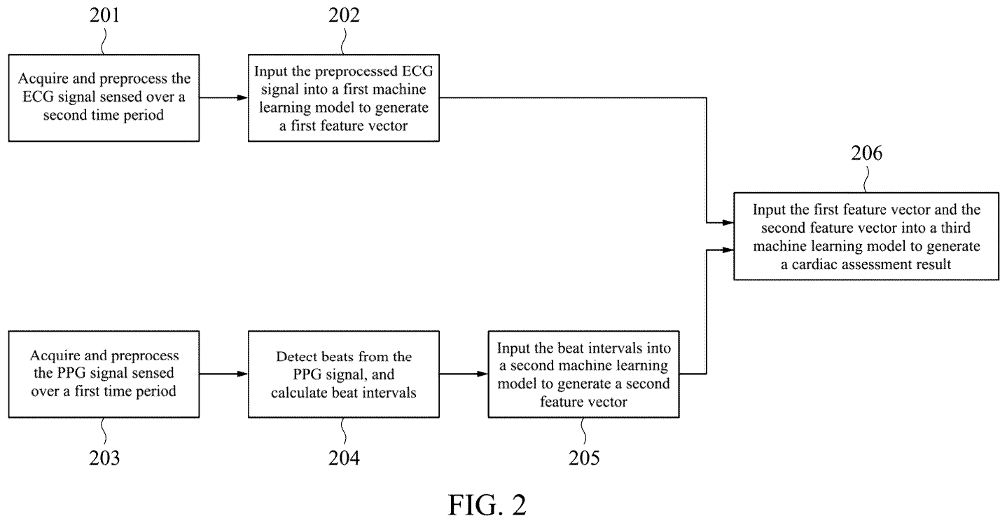

FIG. 2 is a flowchart illustrating the process of generating the cardiac assessment result according to the first embodiment. Referring to FIG. 2, at step 201, the ECG signal sensed over a second time period is acquired and preprocessed. In this embodiment, the second time period is 30 seconds, and the preprocessing may include band-pass filtering, noise removal, and so on. At step 202, the preprocessed ECG signal is input into a first machine learning model to generate a first feature vector. This first machine learning model could be a convolutional neural network, and its architecture could adopt LeNet, AlexNet, VGG, GoogLeNet, ResNet, DenseNet, or YOLO (You Only Look Once), among others. The disclosure is not limited in this regard.

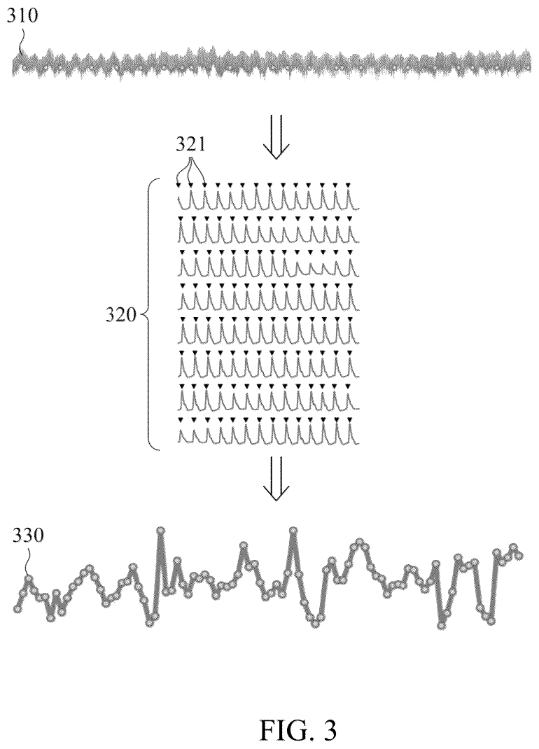

On the other hand, at step 203, the PPG signal sensed over a first time period is acquired and preprocessed. In this embodiment, the first time period is 24 hours, and the preprocessing may include filtering, noise removal, etc. At step 204, multiple beats are detected from the PPG signal, and the beat intervals are calculated. Specifically, the PPG signal reflects changes in blood flow within vessels. When the heart contracts, blood is pumped to various parts of the body, causing a temporary increase in blood flow and pressure within the vessels, affecting the absorption or scattering of light passing through them. Based on the PPG signal, systolic peaks in the vessels can be detected, with each systolic peak representing one beat. A time distance between two adjacent systolic peaks is calculated as the beat interval. In some embodiments, the first time period is divided into multiple sub-periods, and a time segment within each sub-period is selected. Beat intervals are then calculated based on the beats within these selected time segments. For example, FIG. 3 illustrates the processing of the PPG signal according to one embodiment. The PPG signal 310 is continuously sensed over 24 hours. In this case, each sub-period is 1 hour, and the time segment is 5 minutes. Therefore, the PPG signal 310 is divided into 24 sub-signals, and a 5-minute time segment is selected from each. Signal Quality Indices (SQI) can be used to select the 5-minute segment with better signal quality from each hour's PPG signal. As a result, a total of 24 time segments 320 are selected, each containing multiple systolic peaks 321. The time distances between these systolic peaks 321 are the beat intervals. The beat intervals calculated from these time segments 320 are combined to form sequence data 330, where the horizontal direction represents time and the vertical direction represents the magnitude of the beat intervals. It's worth noting that since the time segments are not continuous, two systolic peaks 321 between two time segments are not used to calculate the beat interval. In other embodiments, the aforementioned sub-period could be 30 minutes, two hours, etc., and the length of the time segment could be 1, 2, 3, 4 minutes, etc.

Next, at step 205, the calculated beat intervals (i.e. the sequence data 330), are input into a second machine learning model to generate a second feature vector. This second machine learning model can be, for example, a Transformer, but in other embodiments, it could also be a Recurrent Neural Network (RNN). In some embodiments, it's possible not to divide the PPG signal into multiple sub-signals; instead, the entire PPG signal 310 can be directly input into the second machine learning model to generate the second feature vector.

Finally, at step 206, the first feature vector generated in the step 202 is concatenated with the second feature vector generated in the step 205. This concatenated vector is then input into a third machine learning model to generate the cardiac assessment result. The third machine learning model could be a Multilayer Perceptron (MLP) network, fully connected layers, or similar architectures. In some embodiments, the third machine learning model may also include a softmax function among others. Additionally, in some embodiments, personal information about the user 140 can also be input into the third machine learning model. This personal data may include age, gender, weight, height, medical history, and so on, but the present disclosure is not limited to these.

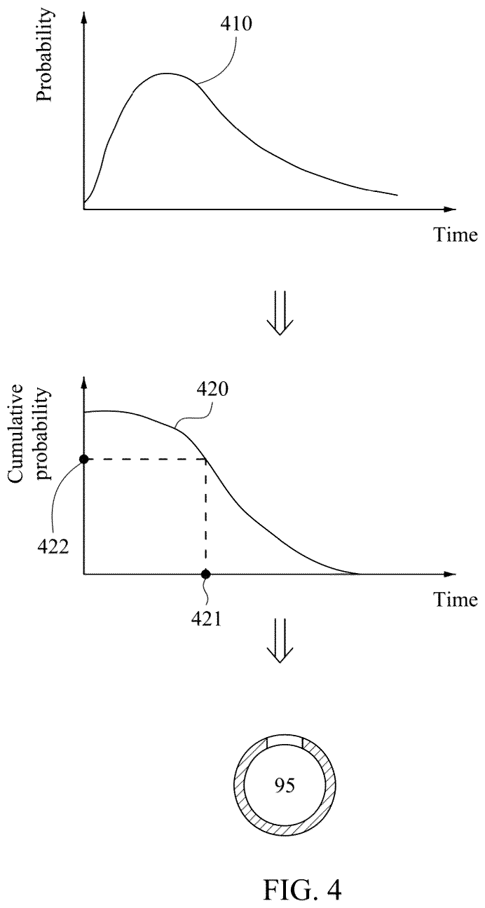

FIG. 4 is a schematic diagram illustrating the cardiac assessment result according to the first embodiment. In this embodiment, the output of the third machine learning model is a vector, where each element in the vector represents a probability of experiencing heart failure at a specific future time, as shown by a curve 410 in FIG. 4. The horizontal axis represents time, while the vertical axis represents the probability of heart failure. Subtracting the probability of heart failure from 1 gives the probability of not experiencing heart failure. Multiplying these probabilities over time yields the cumulative probability of not experiencing heart failure at any point in the future, referred to here as the survival curve 420. The horizontal axis is time, and the vertical axis is the cumulative probability. In other words, the survival curve 420 comprises multiple probabilities of not experiencing heart failure on the time axis. If a predetermined time 421 (e.g., 5 years) is selected on the time axis, the corresponding probability 422 indicates the likelihood that the user will survive for 5 years without experiencing heart failure. Based on the probability 422, a risk score can be generated, such as the risk score “95” shown in FIG. 4, where the probability 422 is positively correlated with the risk score. Various linear or non-linear functions can be used to convert the probability 422 into the risk score. Alternatively, the risk score can be categorized into four groups: Good, Average, Mildly Dangerous, and Severely Dangerous. When the probability 422 exceeds 0.8, the corresponding risk score is Good; between 0.64 and 0.8, it is Average; between 0.44 and 0.64, it is Mildly Dangerous; and below 0.44, it is Severely Dangerous. The above methodology is merely illustrative; the disclosed risk score can be a numerical value, text, a specific pattern, or color, and is not limited thereto.

### Second Embodiment

In the aforementioned first embodiment, a machine learning model is used to extract a feature vector from the signal. In contrast, in the second embodiment, the feature vector is first calculated and then inputted into the machine learning model.

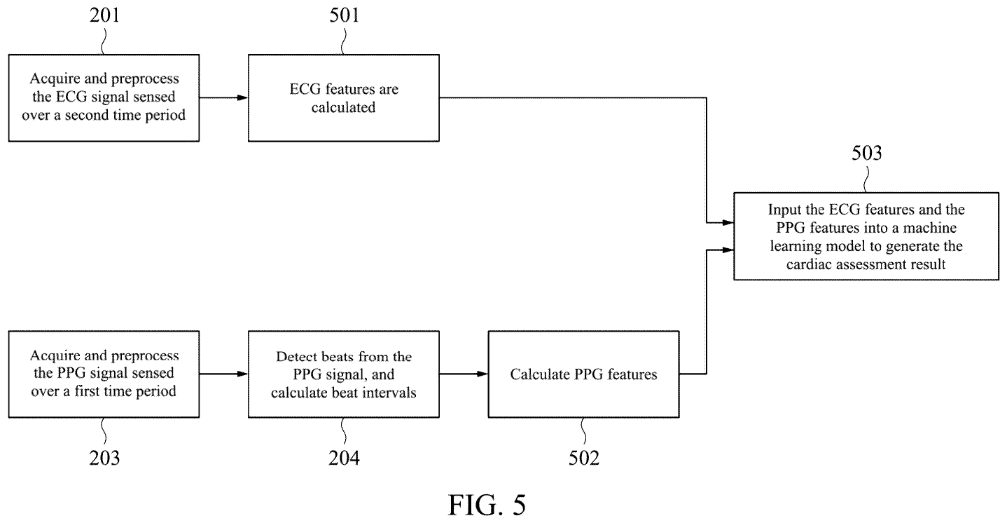

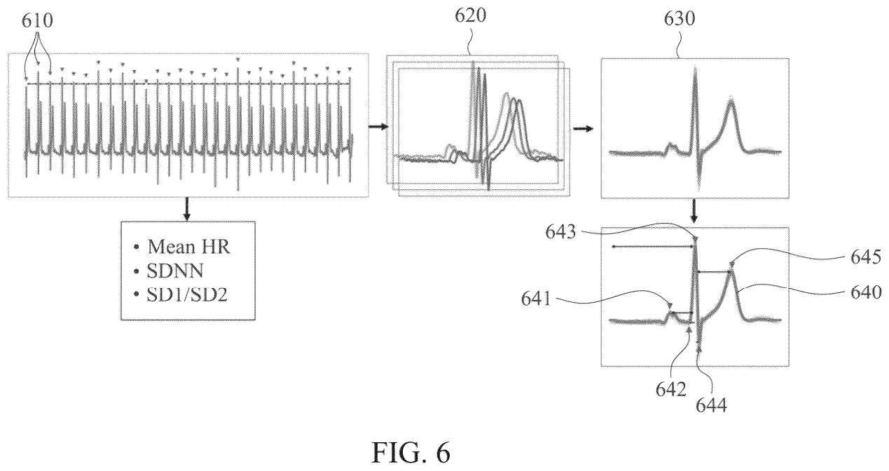

FIG. 5 is a flowchart illustrating the generation of a cardiac assessment result according to the second embodiment. Please refer to FIG. 5, where steps 201, 203, and 204 are the same as those in the first embodiment and will not be elaborated upon here. In step 501, ECG features are calculated. FIG. 6 is a schematic diagram illustrating the calculation of the ECG features according to the second embodiment. In the ECG signal, a single beat includes a P peak, a Q peak, a R peak, a S peak, and a T peak. First, multiple R peaks 610 are detected in the ECG signal. This can be accomplished using any known algorithm, such as by determining if the amplitude of the ECG signal exceeds a certain threshold; the present disclosure is not limited in this regard. Next, the time intervals between these R peaks are calculated, hereinafter referred to as RR intervals. Since one R peak represents one beat, an RR interval also represents the time of one beat. Subsequently, multiple ECG features can be calculated based on these RR intervals. For instance, the average heart rate can be calculated using Equation 1, as described below.

$$MEAN\_HR = \frac{{\sum }_{i = 1}^{K} 60 / RRI [ i ]}{K} \qquad [ Equation 1 ]$$

Where MEAN_HRR represents the average heart rate, i is a positive integer, RRI [i] represents the i^{th} RR interval, and K is the total number of the RR intervals. Additionally, the standard deviation of the RR intervals can be calculated using Equation 2, as described below.

$$SDNN = \sqrt{\frac{{\sum }_{i = 1}^{K} {( RRI [ i ] - \overline{RRI} )}^{2}}{K}} \qquad [ Equation 2 ]$$

Where SDNN represents the standard deviation, and R̅R̅I̅ is the expected value of all RR intervals. In some embodiments, the difference between adjacent RR intervals can also be calculated, represented as ΔRRI [i]. Then, two standard deviations SD1 and SD2 can be calculated using Equations 3-5, where Δ̅R̅R̅I̅ is the expected value of the differences ΔRRI [i]. The ratio SD1/SD2 can serve as an ECG feature.

$$SDSD = \sqrt{\frac{1}{K - 1} {\sum }_{i = 0}^{K} {( Δ RRI [ i ] - \overline{Δ RRI} )}^{2}} \qquad [ Equation 3 ]$$

$$SD 1 = \sqrt{\frac{1}{2} {SDSD}^{2}} \qquad [ Equation 4 ]$$

$$SD 2 = \sqrt{2 {SDNN}^{2} - \frac{1}{2} {SDSD}^{2}} \qquad [ Equation 5 ]$$

Next, in step 620, cardiac cycles in the ECG signal are recognized. For example, a midpoint between two adjacent R peaks can be used as the boundary between two beats. Alternatively, a cardiac cycle can be obtained by extending a certain distance forward and backward from a R peak; the present disclosure is not limited in this regard. Here, each cardiac cycle undergoes padding to ensure that all cardiac cycles have the same length.

In step 630, the aforementioned cardiac cycles are aligned based on their R peaks, and a median is taken to produce a template cycle 640. For example, the time points with the highest amplitude in the R peaks can be aligned, and the median amplitude at each time point can be taken to generate the template cycle 640. Next, a template P peak 641, a template Q peak 642, a template R peak 643, a template S peak 644, and a template T peak 645 are recognized in the template cycle 640. This can be achieved by any known methods. Based on this information, shape features can be calculated. Specifically, a time difference between the template R peak and one of the template P peak, the template Q peak, the template S peak, and the template T peak can be calculated as an ECG feature using Equation 6, as described below.

$${Timing}_{j} = R - j , where j is P , Q , S , T \qquad [ Equation 6 ]$$

Where R represents the time of the template R peak, and j represents the time of the template P peak, the template Q peak, the template S peak, or the template T peak. Timing; represents the time difference.

On the other hand, the amplitude difference between a baseline amplitude and one of the template P peak, the template Q peak, the template R peak, the template S peak, and the template T peak can be calculated as an ECG feature using Equation 7, as described below.

$${Amplitude}_{j} = template [ j ] - baseline , where j is P , Q , R , S , T \qquad [ Equation 7 ]$$

Where Amplitude; represents the amplitude difference, baseline is the baseline amplitude, and template [j] represents the amplitude of the template P peak, the template Q peak, the template R peak, the template S peak, or the template T peak. As for the definition of baseline amplitude, it could, for example, be the median of all amplitudes in the template cycle.

In summary, the aforementioned average heart rate MEAN_HR, the standard deviation SDNN, the ratio SD1/SD2, the time difference Timingⱼ, and the amplitude difference Amplitudeⱼ can all serve as ECG features. In other embodiments, additional ECG features can also be incorporated based on different algorithms; the present disclosure is not limited to these features.

Please refer back to FIG. 5. In step 502, the PPG features are calculated based on the beat intervals. Similar to the first embodiment, in step 204, the beat intervals are generated based on the PPG signal. These beat intervals can be substituted into the aforementioned equations 1 and 2 for RRI [i], thereby calculating the corresponding MEAN_HR and SDNN as the PPG features.

Additionally, the difference between adjacent beat intervals is denoted as ΔBB [i]. Using the following equation 8, the root mean square RMSSD of the successive differences can be calculated as a PPG feature. In this equation, K represents the number of beats in the PPG signal.

$$RMSSD = \sqrt{\frac{1}{K - 1} {\sum }_{i = 0}^{K} Δ {BB [ i ]}^{2}} \qquad [ Equation 8 ]$$

Similarly, by substituting ΔBB [i] into ΔRRI [i] of the equation 3-5, the ratio SD1/SD2 can be calculated as a PPG feature. In some embodiments, π·SD1·SD2 can also be calculated as a PPG feature. In other embodiments, the ratio between the number of consecutive differences ΔBB [i] greater than 50 microseconds and the value K can be calculated, represented as PNN50, as a PPG feature. In some cases, the sample entropy of the PPG signal can also be calculated as a PPG feature.

In step 503, the ECG features calculated in the step 501 and the PPG features calculated in the step 502 are concatenated into a vector and input into a machine learning model to generate the cardiac assessment result. This machine learning model can be a decision tree, random forest, multilayer neural network, convolutional neural network, support vector machine, among others; the disclosure is not limited to these. Moreover, the cardiac assessment result in the second embodiment can be the same as in the first embodiment, including both a survival curve and a risk score. In some embodiments, the machine learning model in step 503 directly outputs a survival curve instead of the curve 410 shown in FIG. 4.

Through the above disclosure, utilizing PPG signals can capture a user's state throughout an entire day, providing comprehensive cardiac information. ECG signals, on the other hand, offer relatively shorter but more accurate cardiac information. The combination of these two types of data produces a better assessment of cardiac status.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A system comprising:
an electrocardiogram (ECG) sensor, configured to obtain an ECG signal related to a user;
a photoplethysmography (PPG) sensor, configured to obtain a PPG signal related to the user; and
a processing circuit, electrically connected to the ECG sensor and the PPG sensor and configured to generate a cardiac assessment result according to the PPG signal sensed during a first time period and the ECG signal sensed during a second time period, wherein the first time period is longer than the second time period.

2. The system of claim 1, wherein the processing circuit is configured to input the ECG signal into a first machine learning model to generate a first feature vector,
wherein the processing circuit is configured to detect a plurality of beats from the PPG signal, calculate a plurality of beat intervals according to the beats, and input the beat intervals into a second machine learning model to generate a second feature vector,
wherein the processing circuit is configured to input the first feature vector and the second feature vector into a third machine learning model to generate the cardiac assessment result.

3. The system of claim 2, wherein the first time period comprises a plurality of sub-periods, the processing circuit is configured to select the beats sensed in a time segment from each of the sub-periods, and calculate the beat intervals according to the selected beats.

4. The system of claim 2, wherein the first machine learning model is a convolutional neural network, the second machine learning model is a transformer, and the third machine learning model is a multilayer perceptron network.

5. The system of claim 1, wherein the processing circuit is configured to recognize a plurality of R peaks in the ECG signal, calculate a plurality of RR intervals among the R peaks, and calculate a plurality of ECG features according to the RR intervals,
wherein the processing circuit is configured to detect a plurality of beats from the PPG signal, calculate a plurality of beat intervals according to the beats, and calculate a plurality of PPG features according to the beat intervals,
wherein the processing circuit is configured to input the ECG features and the PPG features into a machine learning model to generate the cardiac assessment result.

6. The system of claim 5, wherein the processing circuit is configured to recognize a plurality of cardiac cycles from the ECG signal, and align the cardiac cycles based on the R peaks to generate a template cycle,
wherein the processing circuit is configured to recognize a template P peak, a template Q peak, a template R peak, a template S peak, and a template T peak in the template cycle,
wherein the processing circuit is configured to calculate a time difference between the template R peak and one of the template P peak, the template Q peak, the template S peak and the template T peak as one of the ECG features.

7. The system of claim 6, wherein the processing circuit is configured to calculate an amplitude difference between a baseline amplitude and one of the template P peak, the template Q peak, the template R peak, the template S peak, and the template T peak as one of the ECG features.

8. The system of claim 7, wherein the first time period comprises a plurality of sub-periods, the processing circuit is configured to select the beats sensed in a time segment from each of the sub-periods, and concatenate the selected beats to calculate the beat intervals.

9. The system of claim 1, wherein the cardiac assessment result comprises a survival curve and a risk score, and the survival curve comprises a plurality of probabilities of not experiencing a heart failure on a time axis.

10. The system of claim 9, wherein the processing circuit is configured to generate the risk score according to the probability at a predetermined time on the time axis.
